(19) United States
(12) Patent Application Publication
(10) Pub. No.: US 2009/0306482 A1
(43) Pub. Date: Dec. 10, 2009

(54) PATIENT MONITORING SYSTEM AND METHOD

(75) Inventors: Carl Claude Davis, Menomonee Falls, WI (US); Robert Joseph Alberte, JR., Oconomowoc, WI (US); Stephen Thomas Treacy, Menomonee Falls, WI (US)

Correspondence Address: PETER VOGEL;GE HEALTHCARE, 20225 WATER TOWER BLVD., MAIL STOP W492, BROOKFIELD, WI, 53045, US

(73) Assignee: GENERAL ELECTRIC COMPANY, Schenectady, NY (US)

(21) Appl. No.: 12/136,555

(22) Filed: Jun. 10, 2008

(51) Int. Cl. A61B 5/00; G06Q 50/00; G06F 9/46

(52) U.S. Cl. 600/300; 705/2; 718/103

(57) ABSTRACT

A patient monitoring system is disclosed herein. The patient monitoring system includes a processor configured to receive patient data from a monitored patient, to provide a plurality of computer-generated task based on the patient data, and to provide a computer-generated task priority for each of the computer-generated tasks. The patient monitoring system also includes a display connected to the processor. The display is configured to generally simultaneously visually convey the patient data, the computer-generated task, and the computer-generated task priority.

### BACKGROUND OF THE INVENTION

The subject matter disclosed herein relates to a patient monitoring system and method for identifying and managing clinical tasks.

Patient monitoring systems enable a single technician to monitor one or more patients. The patient monitoring systems may, for example, comprise an electrocardiograph, a blood pressure monitor, a thermometer, and/or a pulse oximeter in order to monitor several different physical characteristics of each patient. There are typically a first plurality of tasks related to the physical characteristics being monitored, and a second plurality of tasks related to routine or periodic activities that are performed at regular intervals. These tasks cover a range of importance form low priority to critical.

One problem with conventional monitoring systems is that it is difficult for the technician to systematically identify, prioritize and track all the requisite activities for a given patient. This requirement becomes more problematic in the context of a centralized patient monitoring system wherein a single technician generally simultaneously monitors a large number of patients.

### BRIEF DESCRIPTION OF THE INVENTION

The above-mentioned shortcomings, disadvantages and problems are addressed herein which will be understood by reading and understanding the following specification.

In an embodiment, a patient monitoring system includes a processor configured to receive patient data from a monitored patient, to provide a plurality of computer-generated task based on the patient data, and to provide a computer-generated task priority for each of the computer-generated tasks. The patient monitoring system also includes a display connected to the processor. The display is configured to generally simultaneously visually convey the patient data, the computer-generated task, and the computer-generated task priority.

In another embodiment, a patient monitoring system includes a processor configured to receive patient data from a monitored patient, to provide a plurality of computer-generated tasks based on the patient data, and to provide a computer-generated task priority for each of the computer-generated tasks. The patient monitoring system also includes an input device configured to transmit manually-input tasks and manually-input task priorities. The patient monitoring system also includes a display connected to the processor. The processor is configured to compile the computer-generated tasks and the manually-input tasks in the form of a task queue. The display is configured to generally simultaneously visually convey the patient data and the task queue.

In another embodiment, a method includes collecting patient data from a plurality of monitored patients, implementing a processor to obtain a plurality of computer-generated tasks based on the patient data, and obtaining a computer-generated task priority for each of the computer-generated tasks. The method also includes implementing the processor to generate a task queue comprising the computer-generated tasks, implementing the processor to sequence the computer-generated tasks within the queue based on the computer-generated task priorities, and visually conveying the patient data and the task queue.

Various other features, objects, and advantages of the invention will be made apparent to those skilled in the art from the accompanying drawings and detailed description thereof.

### BRIEF DESCRIPTION OF THE DRAWINGS

FIG. 1 is a schematic representation of a centralized patient monitoring system in accordance with an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

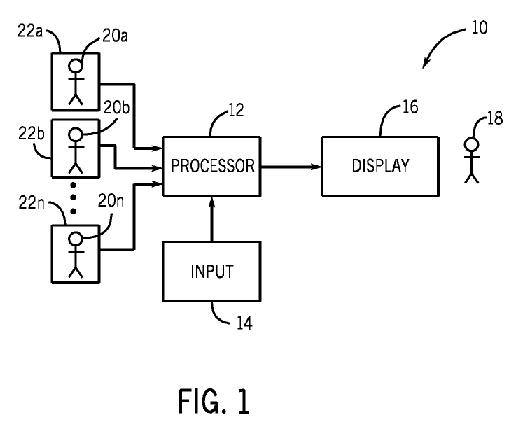

Referring to FIG. 1, a centralized patient monitoring system 10 is shown in accordance with one embodiment. The centralized patient monitoring system 10 includes a processor 12, an input device 14 and a display 16 that are adapted to enable a single technician 18 to generally simultaneously monitor a plurality of patients 20a-20n. While shown and described as components of the centralized patient monitoring system 10, it should be appreciated that the processor 12, the input device 14 and/or the display 16 may be implemented with other patient monitoring systems such as, for example, a single patient bedside monitoring system.

The processor 12 is connected to a plurality of discrete patient monitoring systems 22a-22n. The discrete patient monitoring systems 22a-22n are each configured to monitor one of the patients 20a-20n, to generate patient data based on the specific characteristics being monitored, and to transmit the patient data to the processor 12. In a non-limiting manner, the discrete patient monitoring systems 22a-22n may each comprise an electrocardiograph, a blood pressure monitor, a thermometer, and/or a pulse oximeter. Correspondingly, the patient data generated by the patient monitoring systems 22a-22n may comprise electrocardiogram (ECG) data, blood pressure data, temperature data, and/or pulse data.

The processor 12 is configured provide computer-generated tasks and/or computer-generated task priorities. According to one embodiment, the processor 12 is configured to provide computer-generated tasks and/or computer-generated task priorities based on an evaluation of the patient data from the patient monitoring systems 22a-22n. In a non-limiting manner, the processor 12 may be configured to provide computer-generated tasks and computer-generated task priorities by analyzing the magnitude of the patient data and/or the data's rate of change. As an example, in response to heart rate data falling below a first predetermined value, the processor 12 may provide a computer-generated task comprising the step “alert physician”. The processor 12 may provide a computer-generated task priority based on the degree to which the heart rate data falls below the first predetermined value such that a heart rate falling slightly below the first predetermined value is assigned a “moderate” priority while a heart rate falling drastically below the first predetermined value is assigned a “critical” priority. As another example, in response to body temperature data exceeding a second predefined value, the processor 12 may provide a computer-generated task comprising the step “contact caregiver to administer prescribed medication”. The processor 12 may again provide a computer-generated task priority based on the degree to which the body temperature data exceeds the second predefined value.

According to another embodiment, the processor 12 is configured to provide computer-generated tasks and/or computer-generated task priorities based on an algorithm defining the nature of the task and the associated task priority. As an example, the algorithm may define routine or periodic tasks to be performed at regular intervals. The routine or periodic tasks to be performed at regular intervals may include tasks such as administering prescribed medications, replacing batteries, replacing leads, recording specific types of data, etc. As another example, the algorithm may define a task based on the elapsed time as measured from a preceding task or event. Advantageously, providing computer-generated tasks based on an algorithm allows the technician 18 to track the performance of a variety of routine activities and to coordinate the actions of hospital personnel in order to ensure the activities are efficiently performed. As another example, a computer-generated task priority associated with each of a plurality of different routine tasks may be stored in the form of a database and retrieved by the processor 12.

The input device 14 is connected to the processor 12. In a non-limiting manner, the input device 14 may include a keyboard, mouse, joystick, touch pad, etc. The input device 14 is configured to transmit manually-input tasks and manually-input task priorities to the processor 12. All task priorities (e.g., including both manually-input task priorities and computer-generated task priorities) may optionally be elevated by the processor 12 after a predetermined amount of time has passed in order to increase the likelihood that the associated tasks get performed in a timely manner. The input device 14 may also be implemented to identify tasks that have already been performed, and to thereby avoid the unintentional reprocessing of a task.

The display 16 is connected to the processor 12. The display 16 is configured to visually convey patient data, tasks, task priorities, and/or other types of supplemental data described in detail hereinafter. While depicted as a single device, it should be appreciated that the display 16 may comprise multiple displays. According to one embodiment, the display 16 comprises one or more displays configured to visually convey the patient data for the monitored patients 20a-20n, and one or more additional displays configured to visually convey the tasks and/or task priorities for each of the monitored patients 20a-20n. Alternatively, the display 16 may comprise a single display partitioned to visually convey patient data, tasks and task priorities.

After the processor 12 obtains one or more tasks and/or task priorities from any of the previously described sources, the processor 12 is configured to compile the tasks into a format that facilitates their visual communication. This compilation is generally adapted to assist the technician 18 and to increase the likelihood that important tasks are readily identifiable. The following will provide several different non-limiting embodiments in which the tasks are compiled by the processor 12.

According to one embodiment, the processor 12 may compile a plurality of tasks in the form of a single queue based on a task priority hierarchy such that the highest priority tasks are displayed at the top of the queue and the lowest priority tasks are displayed at the bottom of the queue. According to another embodiment, the processor 12 may compile a plurality of tasks in the form of two separate queues such that critical or high priority tasks are assigned a first queue, and lower priority tasks are assigned a second queue. According to another embodiment, the processor 12 may compile a plurality of tasks in the form of three separate queues such that critical or high priority tasks are assigned a first queue, lower priority tasks are assigned a second queue, and completed tasks are assigned a third queue. It can be seen that each embodiment enables the technician 18 to readily identify those tasks that are most important.

The processor 12 may optionally provide other types of supplementary data along with the compiled tasks. As an example, supplementary data comprising the time at which a task was initially assigned, the identity or location of the patient, the status of a task (e.g., active or processed), the event on which the task is based, etc. may be combined with each compiled task. According to one embodiment, the supplementary data may comprise an appropriate list of options tailored for a specific event. The subsequent selection of an option from the list may in some cases generate additional tasks. According to another embodiment, the supplementary data may comprise a map directing hospital personnel to a specific patient. According to another embodiment, the supplementary data may comprise more detailed information related to a given task such as, for example, an ECG waveform. The compiled tasks and any supplementary data can be transferred to and visually conveyed by the display 16.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

We claim:

1. A patient monitoring system comprising:
a processor configured to receive patient data from a monitored patient, provide a plurality of computer-generated tasks based on the patient data, and to provide a computer-generated task priority for each of the computer-generated tasks; and
a display connected to the processor, said display configured to generally simultaneously visually convey the patient data, the computer-generated task, and the computer-generated task priority.

2. The patient monitoring system of claim 1, wherein the processor is configured to compile the computer-generated tasks in the form of a task queue.

3. The patient monitoring system of claim 2, wherein the processor is configured to sequence the computer-generated tasks based on the computer-generated task priorities.

4. The patient monitoring system of claim 2, wherein said task queue comprises a plurality of queues, and wherein one of said plurality of queues comprises a high priority queue adapted to exclusively retain any high priority tasks.

5. The patient monitoring system of claim 1, wherein processor is configured to provide a second computer-generated task comprising a routine activity performed at regular intervals, and to provide a second computer-generated task priority associated with the second computer-generated task.

6. The patient monitoring system of claim 2, further comprising an input device configured to transmit a plurality of manually-input tasks and a plurality of manually-input task priorities to the processor.

7. The patient monitoring system of claim 6, wherein said processor is configured to incorporate the manually-input tasks into the task queue, and to sequence the manually-input tasks within the task queue based on the manually-input task priorities.

8. The patient monitoring system of claim 1, wherein said display comprises a first portion configured to exclusively convey the patient data and a second portion configured to exclusively convey the computer-generated task.

9. The patient monitoring system of claim 1, wherein said display comprises a plurality of displays including a first display configured to exclusively convey the patient data and a second display configured to exclusively convey the computer-generated task.

10. A patient monitoring system comprising:
a processor configured to receive patient data from a monitored patient, provide a plurality of computer-generated tasks based on the patient data, and to provide a computer-generated task priority for each of the computer-generated tasks;
an input device connected to the processor, said input device being configured to transmit manually-input tasks and manually-input task priorities, and
a display connected to the processor;
wherein the processor is configured to compile the computer-generated tasks and manually-input tasks in the form of a task queue, and wherein the display is configured to generally simultaneously visually convey the patient data and the task queue.

11. The patient monitoring system of claim 10, wherein the processor is configured to sequence the computer-generated tasks and the manually-input tasks within the task queue based respectively on the computer-generated task priorities and the manually-input task priorities.

12. The patient monitoring system of claim 10, wherein said task queue comprises a plurality of queues, and wherein one of said plurality of queues comprises a high priority queue adapted to exclusively retain any high priority tasks.

13. The patient monitoring system of claim 10, wherein processor is configured to provide a second plurality of computer-generated tasks comprising a routine activity performed at regular intervals.

14. The patient monitoring system of claim 10, wherein said display comprises a first portion configured to exclusively convey the patient data and a second portion configured to exclusively convey the computer-generated task.

15. The patient monitoring system of claim 10, wherein said display comprises a plurality of displays including a first display configured to exclusively convey the patient data and a second display configured to exclusively convey the computer-generated task.

16. A method comprising:
collecting patient data from a plurality of monitored patients;
implementing a processor to obtain a plurality of computer-generated tasks based on the patient data;
obtaining a computer-generated task priority for each of the computer-generated tasks;
implementing the processor to generate a task queue comprising the computer-generated tasks;
implementing the processor to sequence the computer-generated tasks within the queue based on the computer-generated task priorities; and
visually conveying the patient data and the task queue.

17. The method of claim 16, further comprising implementing a processor to obtain a second plurality of computer-generated tasks comprising a routine activity performed at regular intervals.

18. The method of claim 16, further comprising obtaining a plurality of manually-input tasks and a corresponding plurality of manually input task priorities.

19. The method of claim 18, further comprising implementing the processor to incorporate the manually-input tasks into the task queue, and to sequence the manually-input tasks within the task queue based on the manually-input task priorities.

20. The method of claim 16, wherein said visually conveying the patient data and the task queue comprises visually conveying the patient data and the task queue using a single device.
